(19) United States
(12) Patent Application Publication
(10) Pub. No.: US 2022/0362256 A1
(43) Pub. Date: Nov. 17, 2022

(54) DISRUPTION OF VASCULAR SMOOTH MUSCLE RELAXATION BY CARFILZOMIB MAY BE THE PRIMARY REASON FOR CFZ-INDUCED VASCULAR DYSFUNCTION

(71) Applicant: Amgen Inc., Thousand Oaks, CA (US)

(72) Inventors: Vishnu CHINTALGATTU, Thousand Oaks, CA (US); Aditya A. GOEL, Thousand Oaks, CA (US); Christina TEKLE, Thousand Oaks, CA (US)

(21) Appl. No.: 17/771,152

(22) PCT Filed: Oct. 22, 2020
(86) PCT No.: PCT/US2020/056863

§ 371 Date: Apr. 22, 2022

Related U.S. Application Data
(60) Provisional application No. 62/924,616, filed on Oct. 22, 2019.

(51) Int. Cl. A61K 31/5377; A61K 31/197; A61K 31/437; A61K 31/519; A61K 45/06; A61P 9/12

(52) U.S. Cl. CPC A61K 31/5377; A61K 31/197; A61K 31/437; A61K 31/519; A61K 45/06; A61P 9/12

(57) ABSTRACT

Provided herein are methods of treating, inhibiting, reducing, or ameliorating cardiovascular adverse events in a patient caused by administration of carfilzomib by administering to the patient at least one of a soluble guanylyl cyclase (sGC) activator, a PDE5 inhibitor, p38 inhibitor, and/or MAPKAPK-2 inhibitor.

### CROSS-REFERENCE TO RELATED APPLICATION

This application claims the benefit of priority of Provisional U.S. Provisional Patent Application No. 62/924,616, filed Oct. 22, 2019, which is incorporated by reference in its entirety.

### BACKGROUND

Carfilzomib (CFZ) is a second-generation proteasome inhibitor that has significantly improved the survival of patients with relapsed or refractory multiple myeloma. CFZ is considered as a highly efficacious proteasome inhibitor with an acceptable safety profile approved for the treatment of relapsed or refractory multiple myeloma (RRMM). CFZ-based regimens have shown significantly prolonged improvement in the overall survival of patients with RRMM compared with standard regimens in the phase 3 ENDEAVOR and ASPIRE trials.¹⁻⁴ Although low incidence rates of grade≥3 cardiovascular adverse events (CVAEs) were observed across various CFZ trials,⁵ patients treated with CFZ had a notable increase in CVAEs compared with those treated with standard therapies.^{2,4} The reported CVAEs include cardiac failure, dyspnea, and hypertension, with hypertension being the most common grade ≥3 CVAE across phase 1 to 3 trials with more than 2000 CFZ-treated patients with RRMM.⁵ In ENDEAVOR and ASPIRE, a subset of patients with RRMM receiving carfilzomib-dexamethasone and carfilzomib-lenalidomide-dexamethasone regimens experienced hypertension (all grades: 25.9% and 15.8%; grade >3: 9.5% and 5.6%, respectively), cardiac failure (all grades: 8.2% and 6.4%; grade ≥3: 4.8% and 3.8%), and ischemic heart disease (all grades: 2.8% and 5.9%; grade ≥3: 1.7% and 3.3%). Furthermore, after thoroughly examining 60 patients treated with CFZ-based regimens for the presence of underlying cardiac risk factors, the presence of any previously known cardiovascular disease was found to be associated with an increased incidence of cardiac events.⁶ However, cardiac adverse events have rarely led to the discontinuation of treatment or death for patients treated with CFZ compared with those treated with standard therapies.⁵

Because CFZ-associated CVAEs may lead to treatment interruption and dose modification, practical management of these events has been proposed.^{7,8} Most recently, an expert panel of the European Myeloma Network and the Italian Society of Arterial Hypertension with the endorsement of the European Hematology Association recommended measures to prevent and manage CVAEs in patients receiving CFZ.⁹ Hypertension, which is the most frequent CVAE among patients treated with CFZ, is also a well-known and potent risk factor for cardiac event onset, including heart failure and ischemic heart disease.⁹ As the first step, the expert panel recommended identifying patients at increased risk for CVAEs by carefully assessing cardiovascular risk factors and prior cardiovascular diseases.

To this day, the molecular mechanism of the CFZ effect on cardiovascular mechanics remains poorly understood.^{10,11} A couple of recent studies using animal models attempted to elucidate how CFZ negatively impacts cardiovascular mechanics. In one study, using isolated rabbit hearts and aortic strips, CFZ increased coronary perfusion pressure, resting vascular tone, and spasmogenic effects, and sharply reduced the vasodilating effect induced by acetylcholine (ACh).¹² In another study, depending on the dosage and administration schedule, CFZ was shown to inhibit AMPKα/mTORC1 pathways through the upregulation of PP2A activity and inactivation of the PI3K/Akt/eNOS (phosphoinositide 3-kinase/Akt/endothelial nitric oxide synthase) pathway in mice.¹³ The co-administration of metformin, an oral hypoglycemic drug used for the management of glucose levels in patients with type two diabetes mellitus, appeared to counteract CFZ-induced cardiotoxicity by restoring the proper regulation of these pathways.¹³ CFZ-induced hypertension has been speculated to be the result of dysregulation in vasoconstriction and vasorelaxation.^{10,11}

### BRIEF DESCRIPTION OF THE FIGURES

FIG. 1A shows that the percent contraction of aortic rings pre-treated with CFZ is similar to that with vehicle control, indicating that CFZ does not affect vascular tone.

FIG. 1B shows that upon inhibiting eNOS that limited NO availability, a significant but minor increase of percent contraction was observed in the aortic rings that were pre-incubated with CFZ.

FIG. 1C shows that no significant CFZ effect on vasoconstriction was observed in the presence of vasopressor—phenylephrine (PE).

FIG. 1D shows that no significant CFZ effect on vasoconstriction was observed in the presence of vasopressor—potassium chloride (KCl).

FIG. 2A illustrates the relaxation of Ach-induced aortic rings was significantly compromised under CFZ treatment as the increased concentration of CFZ resulted in a decrease in percent relaxation at high concentrations of Ach.

FIG. 2B illustrates SNP that provided exogenous NO for VSM did not mitigate the deficiency of vascular relaxation that was prompted by the various amounts of CFZ.

FIG. 2C shows that an increasing concentration of nifedipine causes the plateau of vascular relaxation to be reached at around 50%.

FIG. 3A illustrates the increasing level of phosphorylated eNOS corresponded to the increasing CFZ concentration as the protein expression levels of total eNOS and the housekeeping β3-actin remained the same.

FIG. 3B illustrates that about 30% to 40% increase of NO was observed in the CFZ treated samples when compared with the vehicle control.

FIG. 4 shows induction of soluble guanylyl cyclase (sGC) by carfilzomib (CFZ) in vascular smooth muscle.

FIG. 4A shows Western blot using antibodies specific to sGC-β showed that sGC-β level increased with an increasing amount of CFZ.

FIG. 4B illustrates the gene expression of an sGC subunit, GUCY1A3, was upregulated more than 25 folds after a 24-hour treatment of CFZ in SMCs.

FIG. 4C illustrates that CFZ could not interfere with VSM relaxation in the presence of either sGC activators, cinaciguat or riociguat.

FIG. 5A illustrates sildenafil or tadalafil reversed the CFZ effect on VSM relaxation in the presence of SNP.

FIG. 5B illustrates sildenafil or tadalafil reversed the CFZ effect on VSM relaxation in the presence of ACh.

FIG. 6 illustrates the observation of hypo-phosphorylation of VASP draws a similarity to a recent study on endothelial-independent SMC relaxation.

FIG. 6A illustrates CFZ treatment resulted in a reduced level of p-VASP compared with the vehicle control.

FIG. 6B illustrates protein expression levels of p-VASP assessed by ELISA.

FIG. 7 shows the baseline effect of CFZ on aortic protein activation when aortic rings were incubated with and without endothelium dependent relaxing factor

FIG. 7A shows CFZ caused activation of the P38 pathway with endothelium dependent relaxing factors.

FIG. 7A shows CFZ caused activation of the P38 pathway without endothelium dependent relaxing factors.

FIG. 8 shows the effect of P38 inhibition on CFZ induced vascular dysfunction.

FIG. 8A illustrates SB203580 (p38 inhibitor) prevented CFZ induced vascular dysfunction in the presence of ACh.

FIG. 8A illustrates SB203580 (p38 inhibitor) prevented CFZ induced vascular dysfunction in the presence of SNP.

FIG. 9 shows the effect of MAPKAPK-2 Inhibition on CFZ Induced Vascular Dysfunction.

FIG. 9A illustrates PF3644022 (a MAPKAPK-2 inhibitor) prevented CFZ induced vascular dysfunction in the presence of ACh.

FIG. 9A illustrates PF3644022 (a MAPKAPK-2 inhibitor) prevented CFZ induced vascular dysfunction in the presence of SNP.

FIG. 10 shows the effect of CFZ on vascular tone of isolated rat aortic rings and that CFZ induced increase in vascular tone compared to vehicle that was completely prevented by P38 inhibitor SB203580.

FIG. 11 shows that area under the curve (AUC) of the contraction indicated that CFZ increased angiotensin induced contraction which was prevented by P38 or MAPKAPK-2 inhibition.

### DETAILED DESCRIPTION

Some patients receiving carfilzomib (CFZ) have had an increased incidence of cardiovascular adverse events including hypertension, cardiac failure, and dyspnea but the mechanism of how CFZ induces such cardiovascular dysfunction is poorly understood. There is a need to find methods of treating, inhibiting, reducing, or ameliorating cardiovascular adverse events caused by administration of carfilzomib in patients in need thereof. The inventors have discovered that CFZ induces vascular dysfunction by impairing the mechanism of vascular smooth muscle (VSM) relaxation despite an increase in NO production by the endothelium. With this mechanistic discovery, disclosed herein are compounds that useful in therapies for modulating the signaling components of this mechanism.

In view of the above discovery, described herein are compounds for use in a therapy for treating, inhibiting, reducing, or ameliorating cardiovascular adverse events in a patient being treated with carfilzomib. In some embodiments, the compounds for uses described herein rescue the loss of relaxation of endothelium or vascular smooth muscle cells. In some embodiments, the compounds for such uses are sGC (soluble guanylate cyclase) activators, P38 (mitogen activated protein kinase P38 ) inhibitors, MAPKAPK-2 (mitogen-activated protein kinase-activated protein kinase 2 or MK2) inhibitors, and/or PDE5 (phosphodiesterase 5A) inhibitors.

Examples of sGC activators include BAY 58-2667 (cinaciguat), BAY 63-2521 (riociguat), BAY 60-2770, S-3448, HMR-1766 (ataciguat), or pharmaceutically acceptable salts thereof. In some embodiments, the sGC activator is cinaciguat, riociguat, or pharmaceutically acceptable salts thereof. In some embodiments, the use of sGC activators is in combination with a second compound that is a PDE5 inhibitor, a P38 inhibitor, and/or MAPKAPK-2 inhibitor. Examples of PDE5 inhibitors include sildenafil, tadalafil, avanafil, vardenafil, phentolamine, yohimbine, L-arginine, or pharmaceutically acceptable salts thereof. In some embodiments, the PDE5 inhibitor is tadalafil, sildenafil, or pharmaceutically acceptable salts thereof. Examples of P38 inhibitors include SB 202190, SB 203580, neflamapimod, ARRY371797, PF-06802861, PF 07265803, ralimetinib, LY2228820, or pharmaceutically acceptable salts thereof MAPKAPK2 activation happens downstream of P38 activation. Examples of MAPKAPK2 inhibitor is PF3644022, MK2-IN-1, MK2-IN-1 hydrochloride, MK-2 Inhibitor III, CMPD1, or pharmaceutically acceptable salts thereof. Any one of the compounds or salts thereof described herein can be administered in concurrently (combination) with, prior to, or subsequent to administration of carfilzomib.

In some embodiments, any of the compounds or pharmaceutically acceptable salts thereof described herein are included in a pharmaceutical composition comprising the compound and at least one pharmaceutically acceptable excipients. The pharmaceutical compositions comprising the compounds or salts thereof are useful in a therapy for treating, inhibiting, reducing, or ameliorating cardiovascular adverse events in a patient being treated with carfilzomib. The pharmaceutically acceptable excipients may be chosen from adjuvants and vehicles. The at least one pharmaceutically acceptable excipients, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 19884999, Marcel Dekker, New York discloses various earners used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure.

An “effective amount” of a compound herein, a pharmaceutically acceptable salt thereof, or a pharmaceutical composition thereof is that amount effective for treating, inhibiting, reducing, or ameliorating cardiovascular adverse events listed herein. The exact amount required will vary from subject to subject, depending on the species, age, and general condition of the subject, the severity of the condition, the particular agent, its mode of administration, and the like. That effective amount will vary from patient to patient, depending on the species, age, and general condition of the subject, the severity of the cardiovascular adverse event, the particular active compound, its mode of administration, and the like. It will be understood that the total daily usage of active compound of this disclosure will be decided by the attending physician within the scope of sound medical judgment. The specific effective dose level for any particular patient will depend upon a variety of factors including the disorder being treated and the severity of the disorder; the activity of the specific API employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, such as a mammal, and even further such as a human.

Some compounds, salts, and compositions described herein are marketed drug products and their approved dosages and methods of administration are available on their prescribing information. The compounds, salts, and compositions described herein can be formulated in dosage unit form for ease of administration and uniformity of dosage. The expression “dosage unit form” as used herein refers to a physically discrete unit of agent appropriate for the subject to be treated. The compounds, salts thereof, or pharmaceutical compositions thereof can be administered to patients orally, rectally, parenterally, intracisternally, intravaginally, intraperitoneally, topically (as by powders, ointments, or drops), bucally, as an oral or nasal spray, or the like, depending on the compound and severity of the cardiovascular adverse event being treated.

Further provided herein is a method of treating cancer in a patient, the method comprising administering an effective amount of carfilzomib to the patient, wherein the carfilzomib increases the endothelial function by uploading eNOS activity. In some embodiments, the patient has an increased cardiovascular risk. In some embodiments, the administration of the carfilzomib to the patient interfered with the vasorelaxation of the vascular smooth muscle. In some embodiments, the administration of the carfilzomib dysregulates the gene and protein expression of soluble guanylyl cyclase in vascular smooth muscle tissue. In some embodiments, the administration of the carfilzomib to the patent the dysregulates the phosphorylation of vasodilator-stimulated phosphoprotein in vascular smooth muscle. In some embodiments, the administration of the carfilzomib to the patient increased nitric oxide availability by activating endothelial nitic oxide synthase.

Additional embodiments for any of the compound for use in a therapy for treating, inhibiting, reducing, or ameliorating cardiovascular adverse events in a patient being treated with carfilzomib include:
1. A compound for use in a therapy for treating, inhibiting, reducing, or ameliorating cardiovascular adverse events in a patient being treated with carfilzomib.wherein the compound is a soluble guanylyl cyclase (sGC) activator, P38 inhibitor, MAPKAPK-2 inhibitor, and/or PDE5inhibitor.
2. The compound of embodiment 1, wherein the compound is a soluble guanylyl cyclase activator.
3. The compound of embodiment 1, wherein the compound is a P38 inhibitor and/or MAPKAPK-2 inhibitor.
4. The compound of embodiment 1, wherein the compound is a PDE5 inhibitor.
5. The compound of embodiment 2, wherein the therapy further comprises administration of a second compound that is a PDE5 inhibitor, P38 inhibitor, and/or MAPKAPK-2 inhibitor.
6. The compound of embodiment 3, wherein the therapy further comprises administration of a second compound that is a PDE5 inhibitor or sGC activator.
7. The compound for the use of any one of the preceding embodiments, wherein the cardiovascular effect is at least one of hypertension, pulmonary hypertension, cardiac failure, ischemic heart disease, or dyspnea.
8. The compound for the use of any one of the preceding embodiments, wherein the compound is administered prior to, subsequently to, and/or in combination with carfilzomib.
9. The compound for the use of any one of the preceding embodiments, wherein the compound is administered subsequent to the appearance of the cardiovascular adverse event.
10. The compound for the use of any one of the preceding embodiments, wherein the compound is a sGC activator chosen from BAY 58-2667 (cinaciguat), BAY 63-2521 (riociguat), BAY 60-2770, S-3448, HMR-1766 (ataciguat), and pharmaceutically acceptable salts thereof
11. The compound for the use of any one of the preceding embodiments, wherein the compound is a p38 inhibitor chosen from SB 202190, SB 203580, neflamapimod, ARRY371797, PF-06802861, PF 07265803, ralimetinib, LY2228820, and pharmaceutically acceptable salts thereof.
12. The compound for the use of any one of the preceding embodiments, wherein the compound is a PDE5 inhibitor chosen from sildenafil, tadalafil. avanafil. vardenafil, phentolamine, yohirnbine. L-arginine, and pharmaceutically acceptable salts thereof.
13. The compound for the use of any one of the preceding embodiments, wherein the patient has increased risk for cardiovascular adverse events or is predisposed for cardiovascular adverse events.
14. The compound for the use of any one of the preceding embodiments, wherein the patient is a human
15. The compound for the use of any one of the preceding embodiments, wherein the patient has multiple myeloma.
16. The compound for the use of any one of the preceding embodiments, wherein the patient has relapsed or refractory multiple myeloma (RRMM) or newly diagnosed multiple myeloma (NDMM).
17. The compound for the use of any one of the preceding embodiments, wherein the compound in a pharmaceutical composition comprising the compound and pharmaceutically acceptable excipients.

Further described are embodiments for methods of treating, inhibiting, reducing, or ameliorating cardiovascular adverse events in a patient caused by administration of carfilzomib to the patient, including:
18. A method comprising administering to the patient a pharmaceutically effective amount of at least one compound chosen from soluble guanylyl cyclase (sGC) activator, P38 inhibitor, MAPKAPK-2 inhibitor, and/or PDE5 inhibitor.
19. The method of embodiment 18, wherein the compound is a soluble guanylyl cyclase (sGC) activator.
20. The method of embodiment 18, wherein the compound is a p38 inhibitor and/or MAPKAPK-2 inhibitor.
21. The method of embodiment 18, wherein the compound is a PDE5 inhibitor.
22. The method of embodiment 19, wherein the method further comprises administration of a second compound that is a PDE5 inhibitor, P38 inhibitor, and/or MAPKAPK-2 inhibitor.
23. The method of embodiment 20, wherein the method further comprises administration of a PDE5 inhibitor or sGC activator.
24. The method of any one of the preceding embodiment, wherein said cardiovascular effects is at least one of hypertension, pulmonary hypertension, cardiac failure, ischemic heart disease, or dyspnea.
25. The method of any one of the preceding embodiment, wherein soluble guanylyl cyclase (sGC) activator, a PDE5 inhibitor, p38 inhibitor, and/or MAPKAPK-2 inhibitor is administered prior to, subsequently to, and/or in combination with carfilzoinib.
26. The method of any one of the preceding embodiment, wherein the compound is administered subsequent to the appearance of the cardiovascular adverse event.
27. The method of any one of embodiments 18-26, wherein the soluble guanylyl cyclase (sGC) activator is BAY 58-2667 (cinaciguat), BAY 63-2521 (riociguat), BAY 60-2770, S-3448, HMR-1766 (ataciguat), or pharmaceutically acceptable salts thereof.
28. The method of any one of embodiments 18-27, wherein the method further comprises administering to the patient a PDE5 inhibitor, a P38 inhibitor, and/or MAPKAPK-2 inhibitor.
29. The method of any one of embodiments 18-28, wherein the PDE5 inhibitor is sildenafil, tadalafil, avanafil, vardenafil, phentolamine, yohimbine, L-arginine, or pharmaceutically acceptable salts thereof.
30. The method of any one of embodiments 18-29, wherein the sGC activator is BAY 58-2667 (cinaciguat), BAY 63-2521 (riociguat), BAY 60-2770, S-3448, HMR-1766 (ataciguat), or pharmaceutically acceptable salts thereof
31. The method of any one of embodiments 18-30, wherein the p38 inhibitor is SB 202190, SB 203580, neflamapimod, ARRY371797, PF-06802861, PF 07265803, ralimetinib, LY2228820, or pharmaceutically acceptable salts thereof
32. The method of any one of embodiments 18-31, wherein the patient has increased risk for cardiovascular adverse events or is predisposed for cardiovascular adverse events.
33. The method of any one of embodiments 18-32, wherein the patient is a human
34. The method of any one of embodiments 18-33, wherein the patient has multiple myeloma.
35. The method of embodiment 34, wherein the patient has relapsed or refractory multiple myeloma or newly diagnosed multiple myeloma.

### EXAMPLES

The objective of this study was to investigate CFZ-induced dysregulation of key signaling components in vascular function. To elucidate CFZ-induced cardiovascular effects, myograph was used to monitor changes in the function of rings prepared from rat thoracic aorta. Minor but significant contraction occurred in the presence of N(gamma)-nitro-L-arginine methyl ester, and inhibition of endothelial nitric oxide (NO) production. No significant CFZ effect on contraction occurred in the presence of phenylephrine. Instead, CFZ interfered with acetylcholine-/sodium-nitroprusside—induced vasorelaxation. Because CFZ appeared to increase endothelial contraction by upregulating NO synthase activity, CFZ-induced impairment of vasorelaxation might be associated with vascular smooth muscle (VSM) rather than endothelium. Consistent with a VSM-mediated effect, phosphodiesterase 5 (PDES) inhibitors or soluble guanylyl cyclase (sGC) activators negated vascular effects of CFZ. Vasodilator-stimulated phosphoprotein (VASP), a well-known substrate for protein kinase G (PKG), became less active with CFZ, suggesting that PKG may play a role in CFZ-induced vascular dysfunction. We demonstrated that CFZ impaired VSM-induced relaxation despite an increase in NO production by the endothelium. Our findings with PDES inhibitors and sGC activators may inform strategies directed at VSM to partially mitigate the cardiovascular effects of CFZ, and help improve our clinical understanding of the effect of CFZ on cardiovascular function.

Introduction

In the examples provided herein, the effects of CFZ on vascular endothelium and vascular smooth muscle (VSM) were investigated by monitoring the functions of the rat aortic rings with myograph ex vivo. Endothelial cells are the primary source of the muscle relaxant, nitric oxide (NO), leading to smooth muscle relaxation. The activation of protein kinase A (PKA) increases the intracellular calcium level in smooth muscle cells (SMCs), leading to the phosphorylation of myosin light chain (MLC), and the formation of a cross bridge with actin for initiating contraction. In contrast, the activation of protein kinase G (PKG), resulting from activation of soluble guanylyl cyclase (sGC) in the cyclic guanosine monophosphate (cGMP) signaling pathway by NO leads to a decrease in intracellular calcium and the dephosphorylation of MLC, causing relaxation.

The cGMP signaling pathway is one important pathway involved in VSM relaxation. PKG activation leads to the phosphorylation of vasodilator-stimulated phosphoprotein (VASP) at serine 239; phosphorylation disrupts actin polymerization, resulting in a decrease in contractility of SMCs.¹⁴ Because VASP is a preferential substrate for PKG, the phosphorylated VASP (p-VASP) is the most reliable downstream marker for PKG activity and is often used as an indirect measure of PKG activity in cell-signaling studies. Another consequence of PKG activation is the inactivation of the Ras homolog gene family A (RhoA) signaling that is responsible for the increased contraction in VSM.¹⁵ The presence of an elevated level of cGMP and the phosphorylation of phosphodiesterase 5 (PDE5) by PKG together contribute to the hydrolysis of cGMP to GMP, providing an important negative feedback on the cGMP signaling pathway.¹⁵

Here, by examining the effect of CFZ on the endothelium and VSM, the inventors showed that CFZ specifically impaired VSM relaxation while enhancing endothelial function through the upregulation of the enzyme activity of eNOS. CFZ appeared to mostly influence the cGMP signaling pathway in VSM as dysregulation of the signaling components of this pathway, including sGC, PDE5, and VASP, were demonstrated. Importantly, the targeting of these signaling components with sGC activators and PDE5 inhibitors reversed the negative impact of CFZ on VSM relaxation. The proposed molecular mechanism of action adds to the clinical understanding of the effect of CFZ on cardiovascular function.

Methods

These ex vivo animal studies were undertaken in accordance with Institutional Animal Care and Use Committee (IACUC) guidelines and were approved by the IACUC.

Preparation of Rat Aortic Rings in an Oxygenated Isometric Myograph and Assessment of Vascular Function

An isometric myograph (DMT 620M) was used for ex vivo measurement of vascular function. Eight-to 10-week old Sprague Dawley rats (purchased from Charles River Laboratories, Wilmington, Mass.) were first anesthetized with 3% isoflurane and then euthanized using inhaled desflurane. The aortas were isolated in an ice-cold, oxygenated (95% O₂, 5% CO₂) Krebs-Henseleit buffer (118.3 mM NaCl, 4.7 mM KCl, 1.2 mM MgSO₄, 1.2 mM KH₂PO₄, 2.5 mM CaCl₂, 25 mM NaHCO₃, and 11.0 mM glucose; purchased from Sigma-Aldrich, St. Louis, Mo.). The thoracic aorta was carefully cleaned to remove perivascular fat and cut into 2-mm rings that were mounted between two stainless steel wires in the myograph chamber containing 8 mL of Krebs-Henseleit buffer in 95% O₂ and 5% CO₂ at 37° C. First, the rings were equilibrated in the chamber for 20 minutes, followed by a gradual increase of mechanical stretch until a final stretch of 10 mN (Newton) was reached. After an additional incubation with fresh buffer for 30 minutes, the rings were constricted with 8 mL of 80 mM KPSS (43 mM NaCl, 80 mM KCl, 2.5 mM CaCl₂, 1.2 mM MgSO₄, 1.2 mM KH₂PO₄, 25.0 mM NaHCO₃, and 11.0 mM glucose) for 15 to 20 minutes, followed by 3 washes with the Krebs-Henseleit buffer until the force returned to the baseline stretch. The tension of the aortic rings was continuously measured with the Lab Chart 7 data acquisition system (ADlnstruments, Colorado Springs, Colo.),In Some of the experiments the aortic rings were pre-treated with 100 μM N(gamma)-nitro-L-arginine methyl ester (L-NAME; Sigma-Aldrich) and 10 μM indomethacin for 10 minutes prior to the pre-incubation with CFZ (Amgen Inc.) for 2 hours. After an additional incubation with fresh buffer for 30 minutes, the rings were constricted with 8 mL of 80 mM KPSS (43 mM NaCl, 80 mM KCl, 2.5 mM CaC12, 1.2 mM MgSO4, 1.2 mM KH2PO4, 25.0 mM NaHCO_{3, and} 11.0 mM glucose) for 15 to 20 minutes, followed by 3 washes with the Krebs-Henseleit buffer until the force returned to the baseline stretch. The tension of the aortic rings was continuously measured with the Lab Chart 7 data acquisition system (ADlnstruments, Colorado Springs, Colo.).

To assess the effect of CFZ on vascular contraction, the aortic rings were pre-treated with or without CFZ for 2 hours, followed by treatment with an increasing concentration of phenylephrine (PE) from 10 nM to 1 μM. After the maximum contraction was achieved, the aortic rings were washed 3 times until the force returned to baseline. The contraction was continuously measured and plotted as percent contraction normalized to the response of KPSS. For assessment of the effect of CFZ on vascular relaxation, the pre-treated aortic rings were incubated with 1 μM PE for 20 minutes to induce contraction. For experiments involving sildenafil and tadalafil, a 10-minute pre-treatment with 30 nM of these smooth muscle relaxants was done prior to the incubation with CFZ. After reaching a stable contraction, ACh, sodium nitroprusside (SNP), nifedipine, riociguat, or cinaciguat was added to the chamber at an increasing concentration from 100 μM to 10 μM; each stepwise concentration increase was added after the response to the current concentration reached a plateau. The relaxation was continuously measured and plotted as percent relaxation normalized to the response with 1 μM PE.

Cell culture of endothelial cells and SMCs The primary rat aortic endothelial cells were purchased from Cell Biologics (Chicago, IL). Cells were expanded with 3 to 5 passages and seeded at 3×10⁶ cells/mL in 10-cm plates a day prior to a 2-hour CFZ treatment. Fresh culture medium containing 1×10⁻⁶ M ACh (Sigma-Aldrich) replaced the medium containing CFZ. After 30 minutes of ACh treatment, cells were scraped and collected.

To confirm whether results could be replicated in human tissue, human aortic smooth muscle cells (Lonza, Walkersville, Md.) were cultured in SmGM-2™ Smooth Muscle Cell Growth Medium-2 BulletKit™ (Lonza). The cells were maintained at 37° C. in a humidified incubator with 5% CO₂. Cells were seeded at a concentration of 2.5×10⁵ cells/mL of medium. After 1 day of culture, fresh medium with or without 2.7 μM CFZ was added and incubated for 6 or 24 hours.

Nitrate/Nitrite Assay

Protein levels of eNOS were unable to be assessed in rat endothelial cells; therefore, endothelial cells from mice were used. Mouse coronary endothelial cells (Cell Biologics) at passage 7 were seeded at 50,000 cells per well with 200 μL of endothelial cell media (Cell Biologics) in 96-well plates for 24 hours at 37° C. in 5% CO₂. Some cells were pretreated with 100 μM L-NAME for 1 hour before adding 10 μM ACh. Cells were further treated with 0.3, 0.9, or 2.7 μM of CFZ or its vehicle for an additional 48 hours. The supernatant was collected and the amount of NO was assessed following the manufacturer's protocol for the colorimetric Nitric Oxide Assay Kit (Abcam, Cambridge, Mass.).

RNA isolation and RT-PCR

Cell pellets were lysed in Buffer RLT (Qiagen, Germantown, Md.) supplemented with β-mercaptoethanol (Bio-Rad, Hercules, Calilf.). RNA was isolated and purified using QIAcube (Qiagen). The RNA concentration and quality were determined by NanoDrop 8000 spectrophotometer (Thermo Fisher Scientific, Wilmington, Del.). DNA contaminant was removed through DNase I (Promega, Madison, Wis.) digestion prior to quantitative RT-PCR. Quantitative RT-PCR was performed in the QuantStudio 7 Flex RT-PCR system (Thermo Fisher Scientific) with RNA-to-CT 1-Step Kit (Thermo Fisher Scientific) and TaqMan assays (Applied Biosystems, Foster City, Calif.). The TaqMan assays were GUCY1A3 (Hs01015574_ml) and 18S rRNA. Multiplex reactions were run in triplicates with the genes of interest and 18S rRNA normalization control. The average relative quantities were calculated from three separate experiments.

Western Blot

Each cell pellet was lysed in 50 to 70 μL RIPA buffer (Cell Signaling Technology, Danvers, Mass.) with the HaltTM protease and phosphatase inhibitor cocktail (Thermo Fisher Scientific). Lysates were quantitated and loaded onto the Li-COR Western blot system. Antibodies specific to phosphorylated eNOS and eNOS from Thermo Fisher Scientific, anti-Guanylyl Cyclase 131 (ER-19) (Sigma-Aldrich), and β-Actin (Sigma-Aldrich) were diluted 1:1000 for Western blot. Antibodies specific to p-VASP, VASP, and GADPH-HRP were purchased from Cell Signaling Technology.

Enzyme-Linked Immunosorbent Assay (ELISA)

A total of 50,000 human aortic SMCs were seeded per well in a 96-well plate for 24 hours. Plates were washed 3 times with starvation medium (not containing any serum or growth factors) and incubated in starvation medium overnight. Next day, cells were treated with vehicle or 2.7 μM CFZ with or without the 10-minute pre-treatment with tadalafil. Dose-response curve to SNP was generated; the concentration of SNP was diluted from 1000 μM SNP to 0 μM. Cell lysates from the cell lysis with Meso Scale Discovery (MSD; Rockville, Md.) Tris Lysis Buffer were used to measure phosphorylation of VASP. MSD ELISA (L15XA) plates were coated overnight with 10 μg/mL anti-p-VASP (Ser239) antibodies in 25 μL/well, followed by 3 washes with PBS buffer. Plates were blocked for 1 hour with 3% bovine serum albumin followed by 3 washes with MSD wash buffer before samples (30 μL) were added and incubated for 2 hours. After 3 washes with MSD wash buffer, 50 μL of detection antibody D21FH-1 (1:50 dilution) was added at a 1:50 dilution and incubated for 2 hours, followed by 3 washes. Plates were read after the addition of 2× Read Buffer T.

Statistical Analysis

Statistical analysis was undertaken using GraphPad Prism software (GraphPad Software, San Diego, Calif.; version 8.1.2). Analysis of the nitrate/NO production was performed using one-way analysis of variance with P<0.05. Analysis of the percentage contraction or relaxation was done using an unpaired t test with P<0.05.

Involvement of p38 in CFZ Induced Vascular Dysfunction

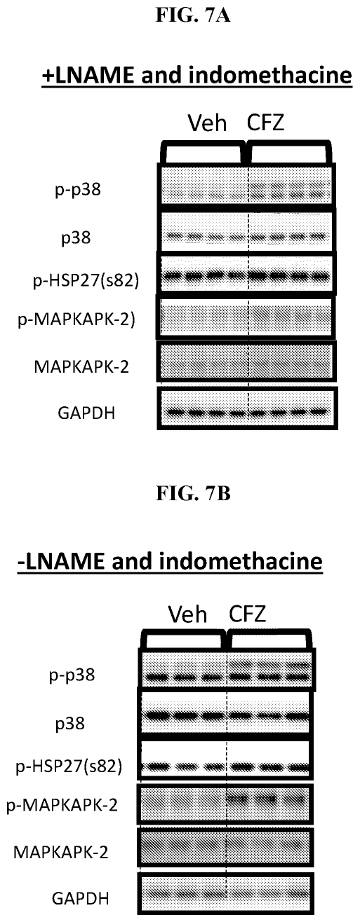

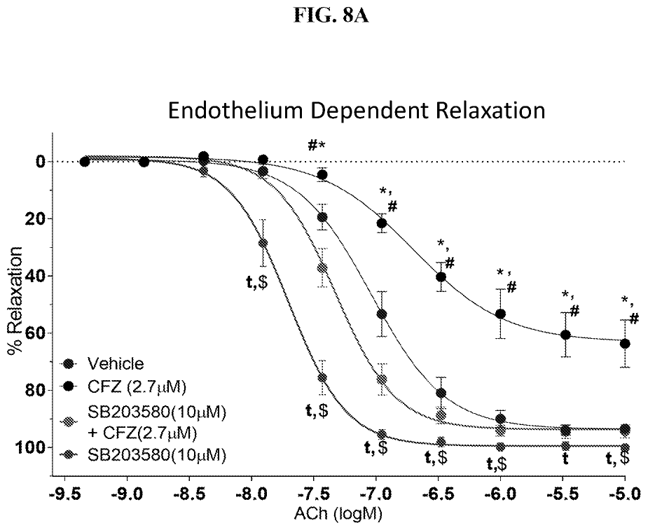

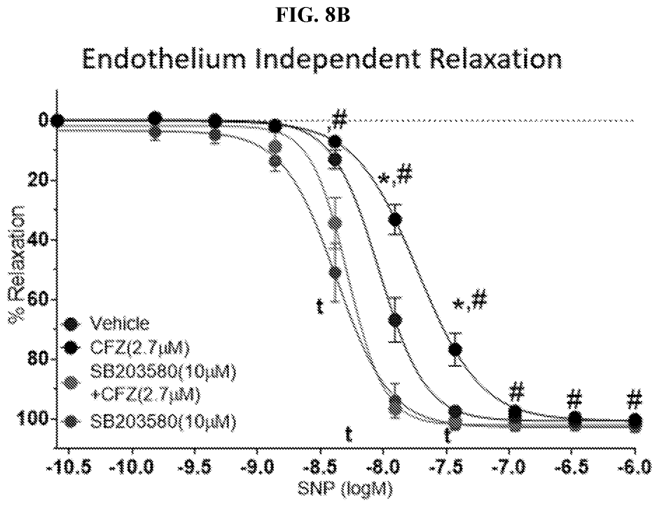

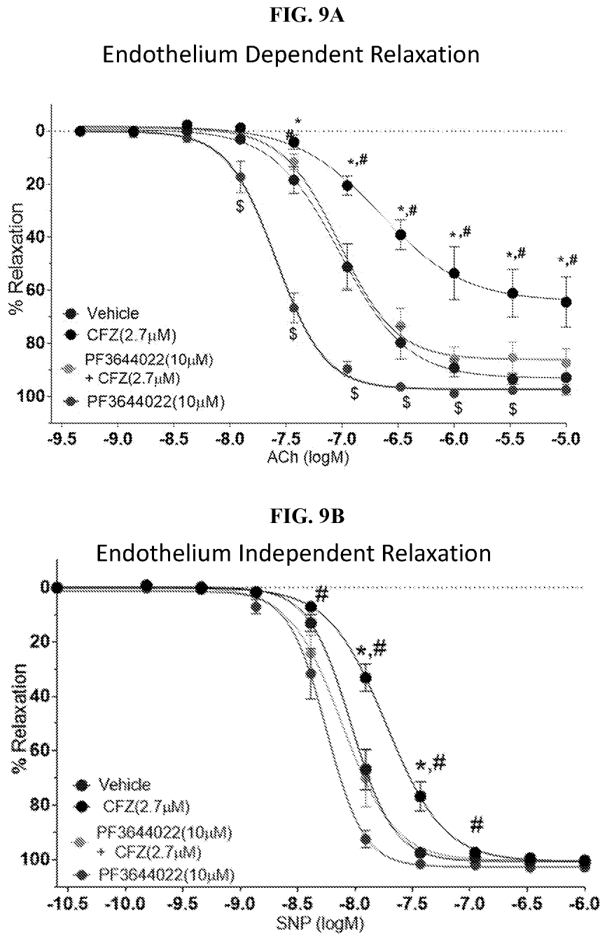

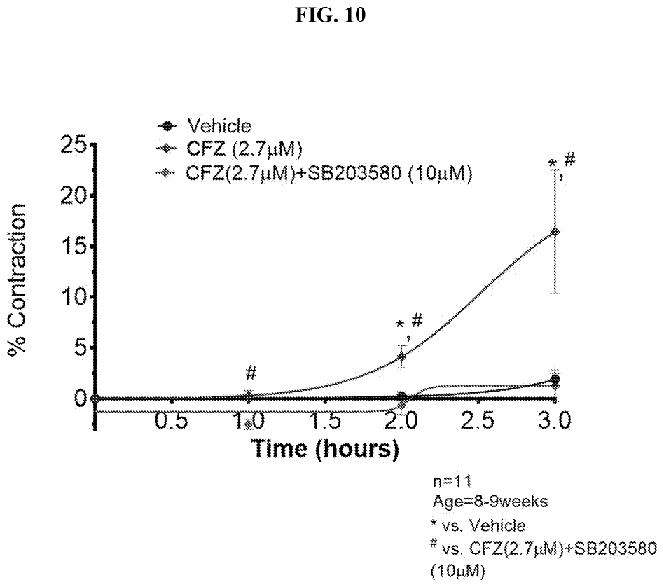

Aortas were isolated from 8-10 week old CD® (Sprague Dawley) rats (from Charles River Laboratories) in an ice-cold, oxygenated (95% O2, 5% CO2) Krebs-Henseleit buffer. The aortas were then carefully cleaned to remove perivascular fat and cut into 2-mm rings that were mounted between two stainless steel wires in the myograph chamber containing 8 mL of Krebs-Henseleit buffer in 95% O₂ and 5% CO₂ at 37° C. Rings were stretch until a final stretch of 10 mN (Newton) was reached and incubated for 30 mins for equilibration. The rings were constricted with 8 mL of80 mM KPSS for 15 minutes, followed by 3 washes with the Krebs-Henseleit buffer until the force returned to the baseline stretch. The aorta were then treated for one of the following experiments:
For protein expression studies the aortic rings were pre-treated with 100 μM N(gamma)-nitro-L-arginine methyl ester (L-NAME; Sigma-Aldrich) and 10 μM indomethacin for 10 minutes prior to the pre-incubation with CFZ (Amgen Inc.) for 1.5 hours. The rings were then isolated for protein expression analysis. Results shown in FIG. 7 and described herein.
For functional vascular studies rings were treated with P38 inhibitor (10 μM, SB203580) or MAPKAPK-2 inhibitor (PF3644022) for 10 mins before the addition of CFZ and incubated for 1 hour followed by contraction induced by Phenylephrine (1 μM) and relxation by Acetylcholine or Sodium Nitroprusside (in presence of endothelium derived relaxing agents blockers). Results are shown in FIGS. 8 and 9 and described herein.
To assess the effect of CFZ on basal tone of aorta, the rings were incubated with 2.7 μM CFZ for 3 hours in presence and absence of 10 μM SB203580. Results shown in FIG. 10 and its description.

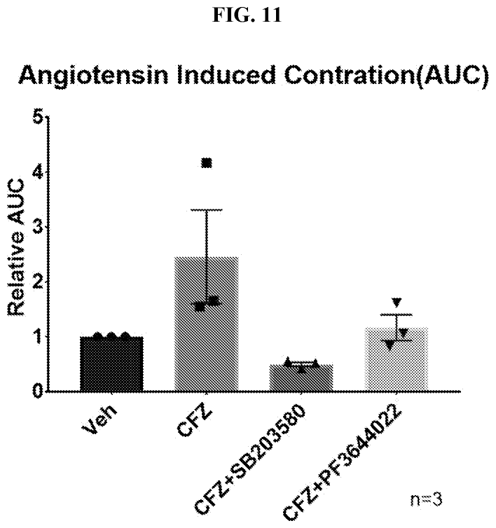

To assess the effect of CFZ and P38 pathway inhibition on angiotensin induced vascular contraction the rings were incubated with 100 μM N(gamma)-nitro-L-arginine methyl ester (L-NAME) and 10 μM indomethacin for 10 minutes, followed by addition of either vehicle or P38 inhibitor (10 μM, SB203580) or MAPKAPK-2 inhibitor (PF3644022) for 10 mins. Following the two incubation the rings were treated with either vehicle or 2.7 μM CFZ for further 1.5 hours before addition of 30nM Angiotensin for 30 min. Angiotensin induced transient contraction which returned to the baseline in 30 mins. Area Under the Curve (AUC) of angiotensin induced contraction was measured. Results are shown in FIG. 11 and described herein.

Results

The attached figures illustrate in detail the following results.

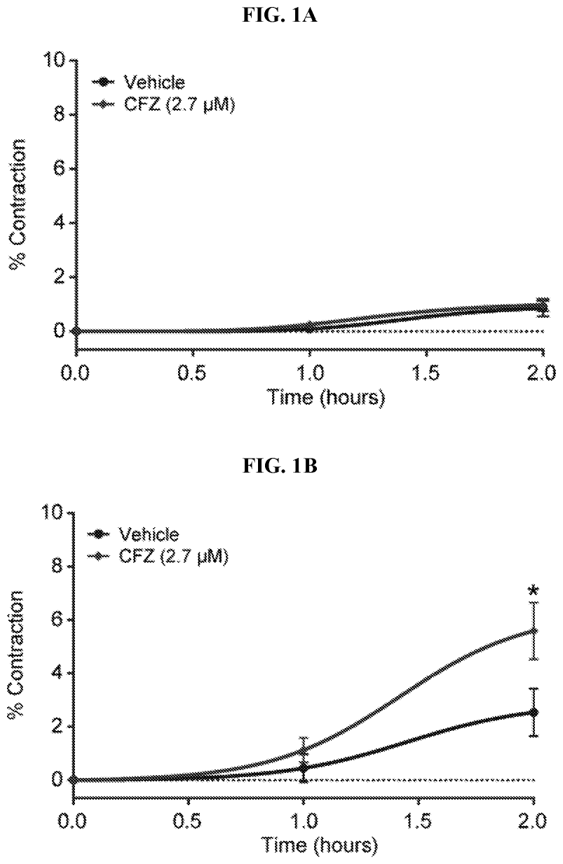

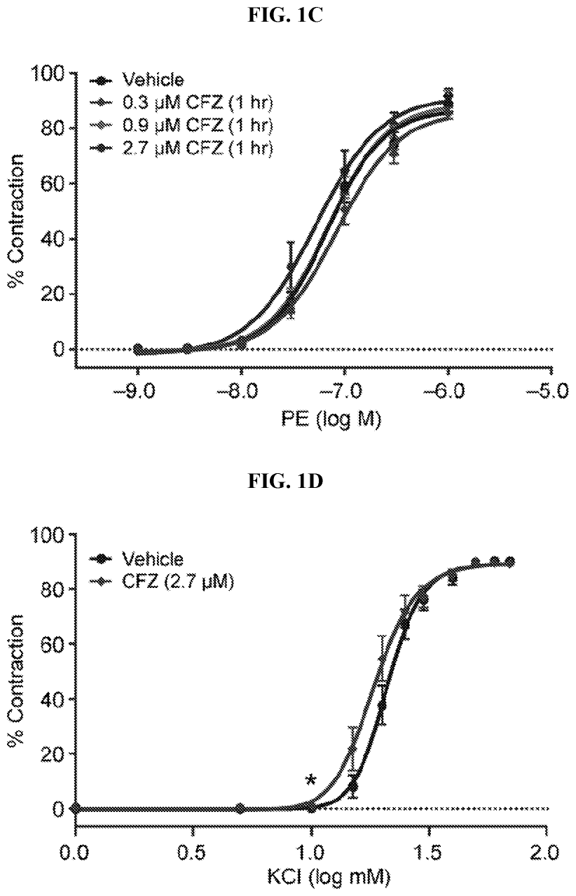

FIG. 1 shows that carfilzomib (CFZ) induces contraction of rat aortic rings treated with L-NAME (NOS inhibitor), but does not interfere with vascular tone or contraction in the presence of vasopressors. FIG. 1A shows the line plot of percent contraction of aortic rings treated with vehicle (blue) or CFZ (red) over time (hours) without L-NAME (n=12). Carfilzomib (CFZ) induces contraction of rat aortic rings treated with L-NAME (NOS inhibitor), but does not interfere with vascular tone or contraction in the presence of vasopressors. FIG. 1B shows the line plot of percent contraction of aortic rings treated with vehicle (blue) or CFZ (red) over time (hours) with 100 μM L-NAME and 10 μM indomethacin for 10 minutes prior to a 2-hour CFZ treatment (n=10). The vertical bars indicate standard deviations. FIG. 1C shows the line plot of percent contraction of aortic rings treated with vehicle (blue) or CFZ at three different concentrations (red, 0.3 μM; green, 0.9 μM; or black, 2.7 μM) that are subjected to an increasing concentration of phenylephrine (log M) (n=10). FIG. 1D shows the line plot of percent contraction of aortic rings treated with vehicle (blue) or CFZ (red) that are subjected to an increasing concentration of potassium chloride (KCl) (log mM) (n=10). The vertical bars indicate standard deviations. The number of measurements is indicated by “n.” * denotes P<0.05.

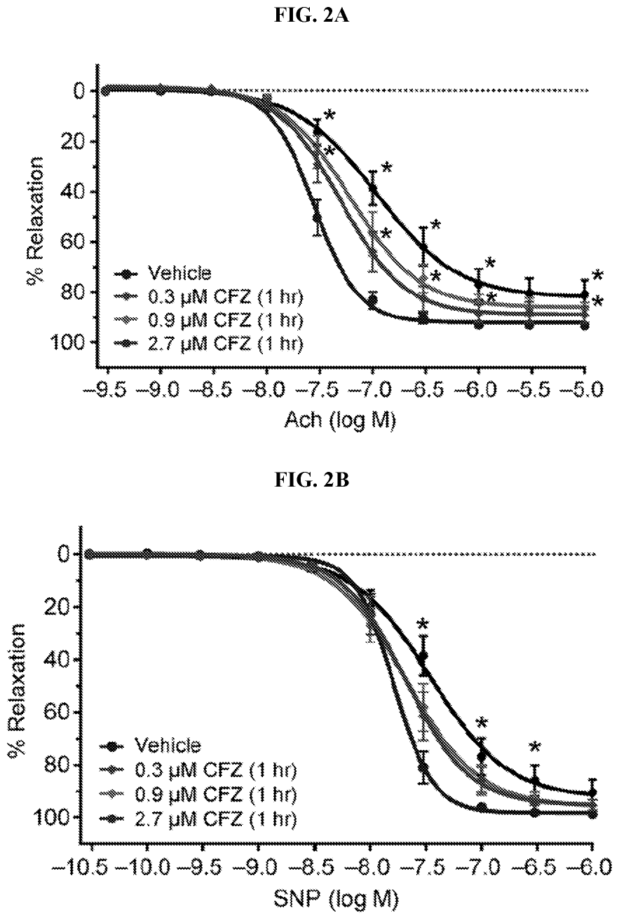

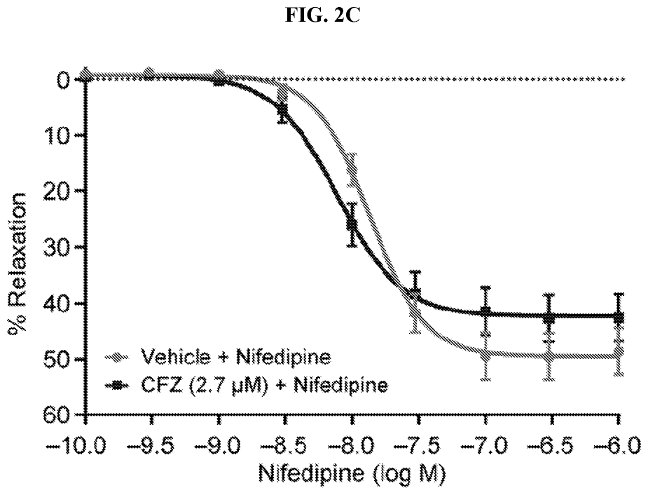

FIG. 2 shows that carfilzomib (CFZ) interferes with vasorelaxation. FIG. 2A shows the line plot of percent relaxation of aortic rings treated with vehicle (blue) or carfilzomib at 0.3 mM (red), 0.9 mM (green), or 2.7 mM (black) that are subjected to an increasing concentration of acetylcholine (ACh) (log M; n=10). * denotes P<0.05; unpaired t-test. Effects of CFZ on aortic rings with two distinct classes of blood pressure lowering drugs. FIG. 2B shows the line plot of percent relaxation of aortic rings pretreated with CFZ in the presence of, sodium nitroprusside (SNP) (log M; n=10). * denotes P<0.05; unpaired t-test. FIG. 2C shows the line plot of percent relaxation of aortic rings pretreated with CFZ in the presence of nifedipine (log M; n=8). The vertical bars indicate standard deviations. The number of measurements is indicated by “n.” * denotes P<0.05; unpaired t-test.

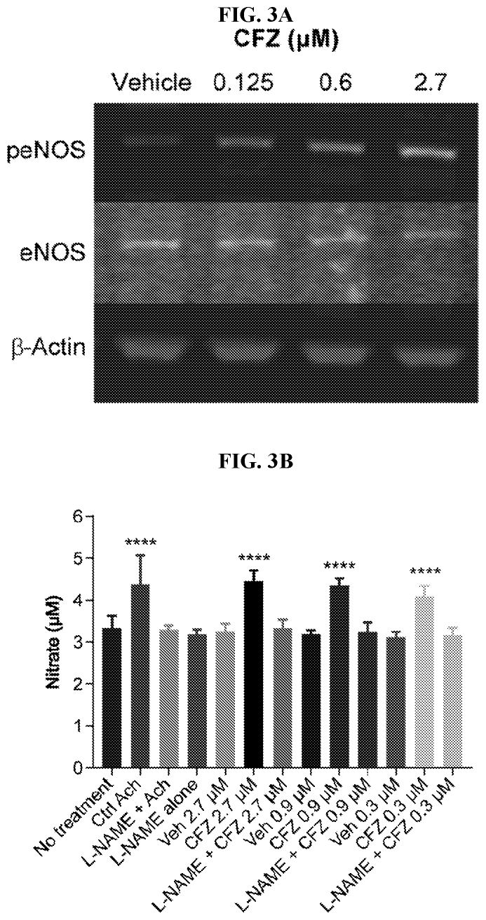

FIG. 3 shows that carfilzomib (CFZ) induces nitrate/nitric oxide production via eNOS activation in mouse endothelial cells. FIG. 3A shows representative fluorescent Western blot showing phosphorylated eNOS (peNOS), eNOS, and b-actin expression in mouse endothelial cells treated with different concentrations of CFZ. FIG. 3B shows nitrate levels in endothelial cells with the indicated treatments on the x-axis of the bar graph (n=8). The number of measurements is indicated by “n.” denotes P<0.0001 (one-way ANOVA). ACh, acetylcholine; Ctrl, control; eNOS, endothelial nitric oxides synthase; peNOS, phosphorylated eNOS; Veh, vehicle.

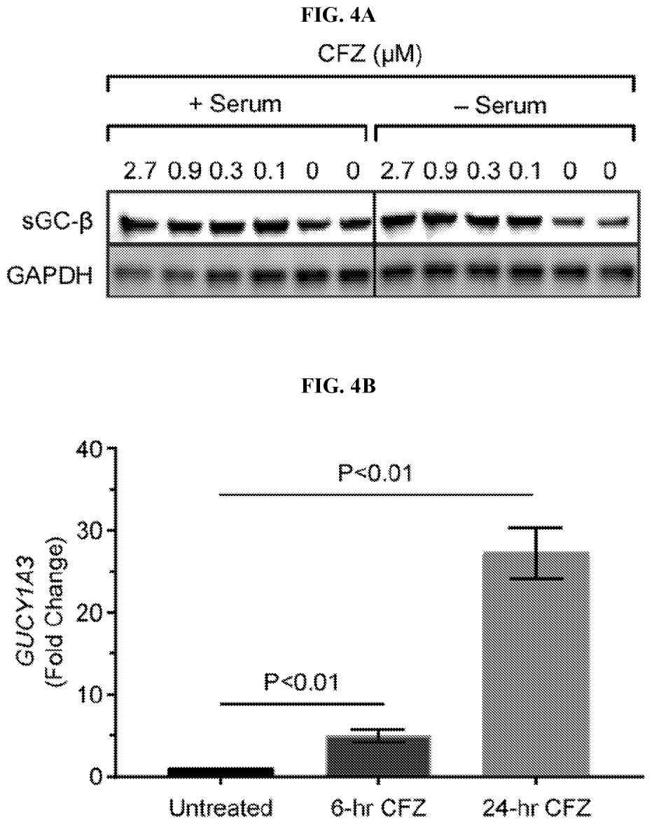

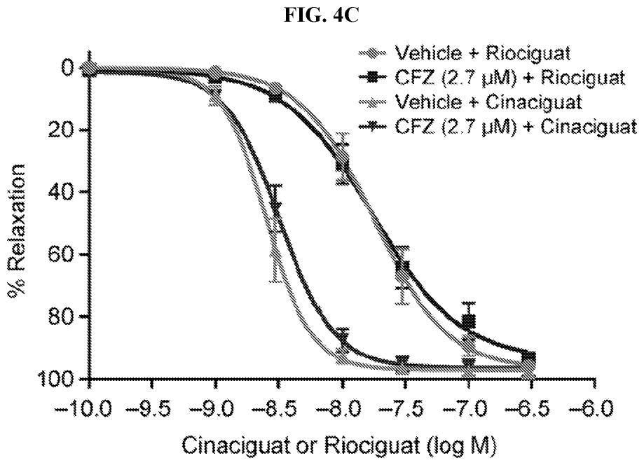

FIG. 4 shows induction of soluble guanylyl cyclase (sGC) by carfilzomib (CFZ) in vascular smooth muscle. FIG. 4A shows western blot showing sGC and GAPDH expression in rat aortic smooth muscle cells treated with CFZ for more than 12 hours. FIG. 4B shows gene expression of GUCY1A3 in human aortic smooth muscle cells upon 6- and 24-hour CFZ treatment (n=3). FIG. 4C shows percent relaxation of aortic rings that are pre-incubated with CFZ or vehicle are treated with an increasing concentration of cinaciguat (log M) or riociguat (log M) (n=8). The number of measurements is indicated by “n.”

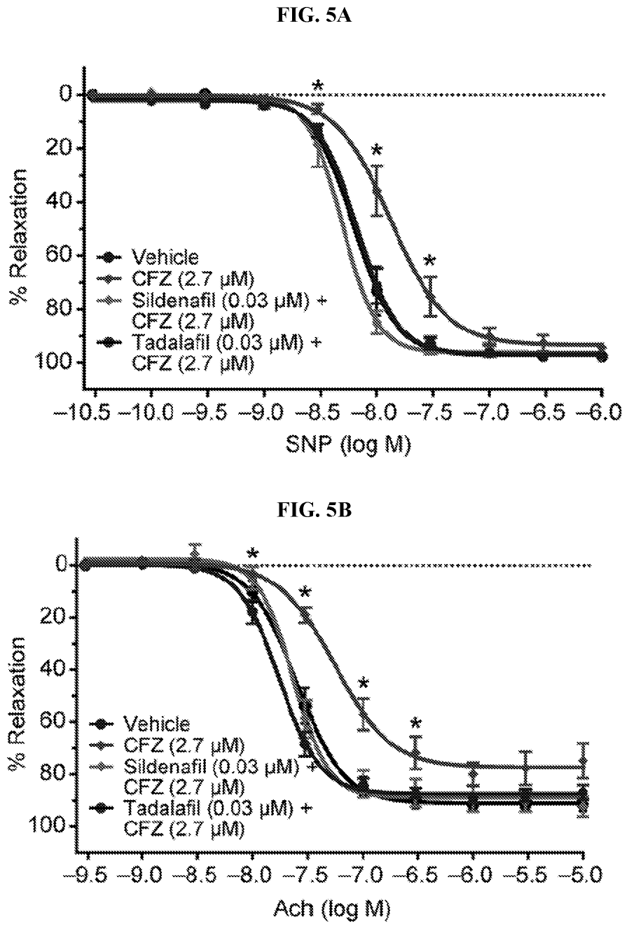

FIG. 5 shows that PDE5 inhibitors counteract the effect of carfilzomib (CFZ) on VSM relaxation (n=8). Aortic rings are pretreated with sildenafil (green) or tadalafil (black) for 10 minutes before the treatment of CFZ (red) or vehicle (blue) with an increasing concentration of FIG. 5A, SNP (log M; n=8) or FIG. 5B, ACh (log M; n=10). * denotes P<0.05 (unpaired t-test). ACh, acetylcholine; PDE5, phosphodiesterase 5; SNP, sodium nitroprusside; VSM, vascular smooth muscle.

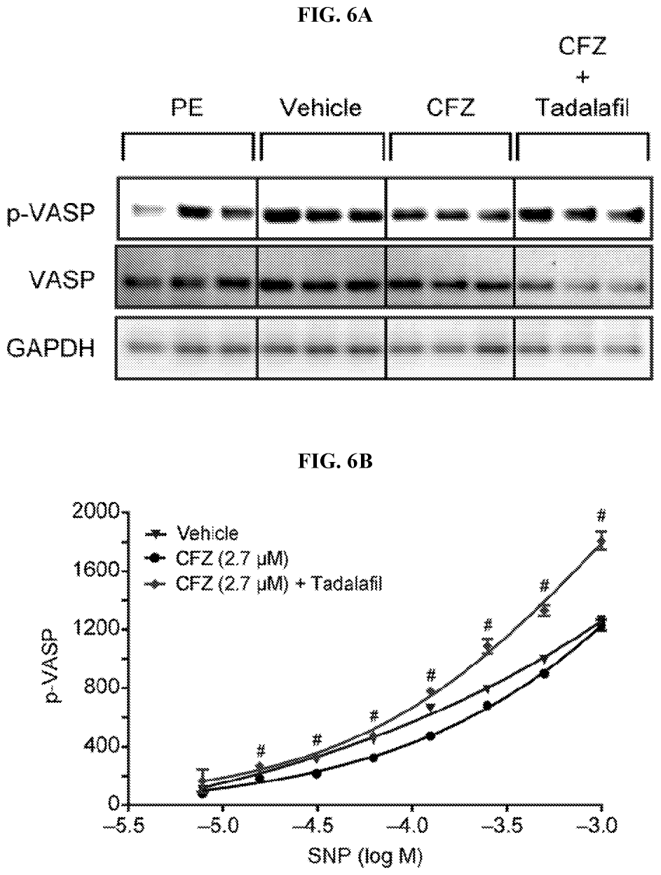

FIG. 6 shows that tadalafil counteracts carfilzomib (CFZ)-induced dephosphorylation of vasodilator-stimulated phosphoprotein (VASP). FIG. 6A shows representative Western blot showing phosphorylated VASP at 5239 (p-VASP), VASP, and GAPDH expression in rat thoracic aortic tissue treated with PE, vehicle, CFZ, or CFZ plus tadalafil. FIG. 6B shows that protein expression levels of p-VASP assessed by ELISA in human aortic smooth muscle cells treated with vehicle (blue), CFZ (black), and CFZ plus tadalafil (red) with an increasing concentration of SNP (log M) (n=4). The number of measurements is indicated by “n.” # denotes P<0.05 (red vs. blue).

FIG. 7 shows the baseline effect of CFZ on aortic protein activation. Aortic rings were incubated for 1.5 hours with or without 2.7 μM CFZ with indomethacine plus LNAME (FIG. 7A) or with indomethacine (FIG. 7B) without 10 min prior addition of endothelium derived relaxing agents blockers. After 1.5 hours of incubation rings were isolate and the protein lysate from them were subjected to western blot assay for activation of p38 pathway. CFZ caused activation of P38 pathway with or without endothelium dependent relaxing factors. Vehicle is indicated with Veh.

FIG. 8 shows the effect of P38 inhibition on CFZ induced vascular dysfunction. Aortic rings were incubated for 10 min with (FIG. 8A) or without (FIG. 8B) endothelium derived relaxing agents blockers for 10 mins followed by addition of SB203580 (p38 inhibitor) in some rings for 10 more mins as indicated. This was followed by addition of 2.7 μM CFZ in some rings as indicated for 1 hour. Rings were then precontracted with 1 μM Phenylephrine followed by relaxation induced by Acetylcholine (FIG. 8A) or Sodium Nitroprusside (FIG. 8B). Incubation with P38 inhibitors prevented CFZ induced vascular dysfunction. *, #, t denotes p <0.05; * is Vehicle vs CFZ; # is CFZ+SB vs CFZ; t is CFZ+SB vs Vehicle; n =10-12, age =8-10 weeks.

FIG. 9 shows the effect of MAPKAPK-2 Inhibition on CFZ Induced Vascular Dysfunction. Aortic rings were incubated for 10 min with (FIG. 9A) or without (FIG. 9B) endothelium derived relaxing agents blockers for 10 mins followed by addition of PF3644022 (MAPKAPK-2 inhibitor) in some rings for 10 more mins as indicated. This was followed by addition of 2.7 μM CFZ in some rings as indicated for 1 hour. Rings were then precontracted with 104 Phenylephrine followed by relaxation induced by Acetylcholine (FIG. 9A) or Sodium Nitroprusside (FIG. 9B). Inhibition of MAPKAPK-2, a downstream effector of P38 , with PF3644022 prevented CFZ induced vascular dysfunction. *, $, # denotes p <0.05; * is Vehicle vs CFZ; # is CFZ+PF3644022 vs CFZ; $ is CFZ+PF3644022 vs PF3644022; n =10-12, age =8-10 weeks.

FIG. 10 shows the effect of CFZ on vascular tone of isolated rat aortic rings. Aortic rings were incubated for 10 min with 100 μM LNAME+10 μM indomethacin (endotheliμM derived relaxing agents blockers) for 10 mins followed by addition of 10 μM SB203580 (p38 inhibitor) in some rings for 10 more mins as indicated. This was followed by addition of 2.7 μM CFZ in some rings as indicated for 3 hour. Contraction was measure at times points indicated and normalized to contraction induced by 80 mM KPSS. CFZ induced increase in vascular tone compared to vehicle that was completely prevented by P38 inhibitor SB203580.

FIG. 11 shows angiotension induced contraction (AIC). Aortic rings were incubated for 10 min with 100 μM LNAME +10 μM indomethacin (endothelium derived relaxing agents blockers) for 10 mins followed by addition of 10 μM SB203580 (p38 inhibitor) or PF3644022 (MAPKAPK-2 inhibitor) in some rings for 10 more mins as indicated. This was followed by addition of 2.7 μM CFZ in some rings as indicated for 1.5 hour. 30 nM Angiotensin was added for 30 min. Angiotensin induced transient contraction which returned to the baseline in 30 mins. Area under the curve of the contraction indicated that CFZ increased angiotensin induced contraction which was prevented by P38 or MAPKAPK-2 inhibition. Vehicle is indicated with Veh.

CFZ Induces Minor Contraction of Rings Prepared from Isolated Rat Thoracic Aorta Under Limited NO Availability but Does Not Affect Vascular Tone

Based on a previous study, CFZ was shown to increase basal vascular tone of thoracic aorta.¹² To investigate the effect of CFZ on the basal vascular tone of aortic rings, the rings were pre-incubated with CFZ, followed by the measurement of vascular function in an oxygenated myograph chamber. The CFZ concentration of 2.7 μM was the highest amount that could be applied, as a further increase of CFZ resulted in a saturated solution and precipitation of CFZ (data not shown). Figure lA shows that the percent contraction of aortic rings pretreated with CFZ is similar to that of rings pretreated with vehicle control, indicating that CFZ alone did not affect vascular tone in vitro in our study. This finding was contradictory to that previously observed.² However, inhibition of eNOS, which subsequently limited NO availability, resulted in a significant but minor increase of percent contraction in aortic rings that were preincubated with CFZ (FIG. 1B). Since endothelial dysfunction is characterized by limited NO availability, these results suggest that CFZ might negatively impact those patients with existing cardiovascular endothelial dysfunction. In those without any endothelial dysfunction, results suggest that CFZ would not have any adverse effect on basal vascular tone.

CFZ Interferes with VSM Relaxation

Hypertension that results from CFZ is likely to be contributed to by dysregulation in endothelium and VSM. To determine how CFZ might affect the normal functions of the endothelium and VSM, the potential impact of CFZ on the vascular relaxation and contraction states of the aortic rings was investigated. No significant CFZ effect on vascular contraction was observed in the presence of vasopressor—phenylephrine (FIG. 1C) or potassium chloride (FIG. 1D); the degrees of contraction observed were similar between CFZ-treated and control aortic rings at increasing levels of vasopressor. For phenylephrine-induced contraction, the effective dose for 50% (EC₅₀) was 8.9×10⁻⁸ with the vehicle alone and 7.7×10⁻⁸ in the presence of CFZ 2.7 μM. The relaxation of ACh-induced aortic rings was significantly compromised under CFZ treatment; increased concentrations of CFZ resulted in a decrease in percent relaxation at high concentrations of ACh (FIG. 2A); the EC₅₀ for ACh-induced relaxation was 3.2×10⁻⁸ with the vehicle alone versus 1.4×10⁻⁷ in the presence of CFZ 2.7 μM. These results suggest that CFZ negatively impacts ACh-induced vascular relaxation, but does not interfere with vasoconstriction.

The experiment above appeared to rule out endothelial dysfunction as a cause for the CFZ-induced defect in cardiovascular relaxation (FIG. 2). Two blood pressure lowering drugs, SNP (an NO donor, causing vasodilation through cGMP/PKG signaling)¹⁶ and nifedipine (decreases intracellular calcium by inhibiting influx of calcium ions thereby inducing smooth muscle relaxation),¹⁷ both function by inducing vasodilation of arterioles and venules. Consistent with an endothelium-independent defect, the presence of SNP, providing exogenous NO for VSM, did not mitigate the reduction in vascular relaxation that was associated with increasing concentrations of CFZ (FIG. 2B). The EC₅₀ for SNP-induced relaxation in the presence of L-NAME and indomethacin was 1.8×10⁻⁸ with the vehicle alone versus 4.3×10⁻⁸ in the presence of CFZ 2.7 μM. These results further suggest that dysregulation of VSM relaxation, rather than endothelial dysfunction, is the likely underlying cause of hypertension associated with CFZ, due to CFZ-induced impairment of VSM relaxation. Unlike SNP, nifedipine relaxes blood vessels by acting as a calcium channel blocker. FIG. 2C shows that an increasing concentration of nifedipine causes vascular relaxation to plateau at around 50%. The pretreatment of aortic rings with CFZ did not appear to significantly affect vascular relaxation in the presence of nifedipine as the responses in both curves were similar (FIG. 2C). These results indicate the distinct mechanism of action of the two blood pressure lowering drugs; CFZ appeared to interfere with SNP-induced relaxation but did not appear to affect nifedipine-induced relaxation.

CFZ-Induced Impairment of Vascular Relaxation is not the Result of Endothelial Dysfunction

CFZ appeared to compromise vascular relaxation (FIG. 2A) by endothelial dysregulation in the presence of limited NO availability and by dysregulation of VSM. NO in endothelial cells is generated by the phosphorylated or active form of eNOS. To check for potential CFZ effect on the protein level and phosphorylation status of eNOS, Western blot with antibodies specific to eNOS and the phosphorylated form of eNOS were used to evaluate the protein expression levels in mouse endothelial cells. The increasing level of phosphorylated eNOS corresponded to the increasing CFZ concentration as the protein expression levels of total eNOS and the housekeeping β-actin remained the same (FIG. 3A). The elevated level of active eNOS suggested that CFZ induced eNOS activity in a dose-dependent manner, which should lead to an increase in NO availability for VSM. An increase in NO of about 30% to 40% was observed in the CFZ-treated samples compared with the vehicle control (FIG. 3B). The concentration of NO in endothelial cells pretreated with CFZ 2.7 μM was similar to that observed in cells pretreated with ACh (FIG. 3B). Contrary to our expectation, CFZ did not cause endothelial dysregulation; rather, it enhanced endothelial function.

The sGC Activators and PDE5 Inhibitors Negate CFZ-Induced VSM Dysregulation

Because CFZ mainly impaired VSM relaxation, the potential dysregulation of one of the signaling components involved in VSM relaxation, the cGMP, was further investigated. During VSM relaxation, the heterodimeric sGC, which is composed of one alpha and one beta subunit, converts guanosine triphosphate into cGMP, activating PKG. The subsequent phosphorylation of downstream molecules by PKG leads to VSM relaxation. The enzyme PDE5 acts as a negative feedback loop for cGMP signaling by breaking the phosphodiester bond of cGMP, thus preventing further PKG activation in VSM. To systematically investigate the potentially dysregulated cGMP signaling components, the protein level of sGC was first assessed. Western blot analysis using antibodies specific to sGC-β showed that the sGC-β level increased with increasing exposure to CFZ (FIG. 4A); it is possible that this occurs as a consequence of irreversible proteasome inhibition by CFZ preventing catabolism of sGC-β.

Interestingly, the gene expression of an sGC subunit, GUCY1A3, was upregulated more than 25-fold after 24-hour treatment of CFZ in SMCs (FIG. 4B), suggesting that sGC subunits were upregulated both at the protein and transcript levels. In accordance with a CFZ-induced dysregulation of sGC, CFZ did not interfere with VSM relaxation in the presence of sGC activators, cinaciguat or riociguat (FIG. 4C); EC₅₀ for relaxation with the sGC activators was similar with or without CFZ (e.g., EC₅₀ for riociguat alone was 1.8×10⁻⁸; for riociguat with CFZ, was 1.7×10⁻⁸). These results, taken together, suggest that CFZ dysregulated sGC levels and that the presence of sGC activators were able to negate the effect of CFZ on VSM relaxation.

Because sGC activation could overcome the CFZ-induced impairment of VSM relaxation (FIG. 4), we hypothesized that the accumulation of the sGC substrate, cGMP, would also negate the effect of CFZ. Known PDE5 inhibitors, sildenafil and tadalafil, prevent the conversion of cGMP to GMP, leading to an accumulation of cGMP Similar to the effect of sGC activators, the PDE5 inhibitors sildenafil or tadalafil reversed the CFZ effect on VSM relaxation in the presence of SNP (FIG. 5A). The EC₅₀ for SNP-induced relaxation for CFZ alone was 1.7×10⁻⁸ versus 5.2×10⁻⁹ for CFZ in combination with sildenafil 30 nM, 6.8×10⁻⁹ in combination with tadalafil 30 nM, and 7.2×10⁻⁹ for vehicle alone. Similarly, the sGC activators reversed the effect of CFZ on VSM relaxation in the presence of ACh (FIG. 5B). The EC₅₀ for CFZ alone was 7.3×10⁻⁸ versus 2.8×10⁻⁸ in combination with either sildenafil 30 nM or tadalafil 30 nM and 1.9×10⁻⁸ for vehicle alone. Furthermore, inhibition of PDE5 in SMCs that leads to the accumulation of cGMP activates PKG, which in turn phosphorylates VASP. Therefore, the phosphorylation status of VASP has been considered as an indication of PKG activity.¹⁸ In line with the lowering of PKG activity, CFZ treatment resulted in a reduced level of p-ASP compared with the vehicle control alone (FIG. 6A). Pre-treatment with tadalafil rescued the CFZ effect by overcompensating for reduced phosphorylation level of VASP (FIG. 6A and B). Taken together, these results suggest that the dysregulation of VSM relaxation is likely caused by the reduced activity of PKG, which negatively impacts the phosphorylation of VASP. The level of p-VASP was restored by co-treatment with PDE5 inhibitors.

Discussion

The purpose of this study was to decipher the mechanism of CFZ-induced hypertension, particularly its influence on the endothelium and VSM that cause vasoconstriction and vasorelaxation. Unlike previous reports,¹² we were unable to observe any significant effect of CFZ on the basal vascular tone of rat aortic rings. In our experiment (FIG. 1A), no detectable changes in the basal vascular tone were observed even with treatment with a saturated solution (i.e., excess) of CFZ. The discrepancy in the results was possibly due to the difference in species being tested (rabbit versus rat) as the experimental designs were similar in both studies. Importantly, we observed a minor yet significant effect of CFZ on basal vascular tone in aortic rings pre-treated with L-NAME (FIG. 1B). The chemical induction with L-NAME mimics endothelial dysfunction and the finding implies that patients who have pre-existing cardiovascular dysfunction may be more susceptible to the risk of CFZ-induced CVAEs than those without cardiovascular dysfunction; although clinical validation of our in vitro results will be required.

When we evaluated the CFZ effect on contraction and relaxation of the aortic rings; CFZ treatment only negatively affected relaxation (FIGS. 1C, 1D and 2). At first, we speculated that the CFZ-induced defect in vasorelaxation was caused by endothelial dysfunction for the following reasons. First, circumstantial evidence has led to the belief that the effect of CFZ is likely endothelial; manifestations related to CFZ treatment include hypertension, a reversible rise in creatinine, a common acute rise in NT-proBNP (N-terminal pro-B-type natriuretic peptide), and lack of evidence for isolated structural cardiomyopathy.¹¹ Second, NO deficiency causes hypertension.¹⁹ Lastly, previous studies have shown that the anti-cancer drug doxorubicin causes endothelial dysfunction by reducing NO production and producing reactive oxygen species that directly damages the endothelium, leading to a defect in vasorelaxation.^{20,21}

However, our in vitro study determined that CFZ treatment did not limit NO availability, instead, it increased NO availability probably by activating endothelial NO synthase (FIG. 3). The increase in NO availability could be a consequence of an innate compensatory mechanism of the endothelium to mitigate the effect of vasorelaxation or it could be the result of a continuous signal that was unable to be switched off properly because of the defect in vasorelaxation. From two recent in vivo studies monitoring patients treated with CFZ-based regimens,^{22,23} reduced dilation of the artery and the excessive stress to the heart appeared to contribute to the defect in cardiovascular mechanics potentially associated with CFZ. For example, a decrease in flow-mediated dilatation of the brachial artery (FMD) that coincided with the insufficient recovery of proteasome activity²² and secretion of B-type natriuretic peptides (BNPs), probably related to excessive stress to the heart, were observed in patients treated with CFZ.²³ The authors of the FMD study assumed the in vivo observations in patients treated with CFZ were suggestive of endothelial dysfunction, even though the molecular mechanism of CFZ is not yet fully understood. Based on our results, we would argue that endothelial dysfunction is unlikely to play a role in CFZ-induced vascular dysfunction as CFZ was observed to stimulate the production of NO in the endothelium. Based on our results, it is suggested that the previous observations can be explained by a CFZ-induced defect in vasorelaxation rather than endothelial dysfunction.

Since CFZ did not interfere with either basal vascular tone or endothelial function (FIG. 1), the most likely explanation for the in vivo observations above would be an endothelium-independent dysregulation of VSM. Because the VSM could not relax properly, the signaling molecules of the major cGMP pathway that regulate relaxation in SMCs were investigated. Based on our observation that both sGC and VASP were dysregulated when compared with normal controls (FIGS. 4A and B, and 6A), we speculated that the inappropriate gene expression and protein levels of sGC and the phosphorylation status of VASP could be restored by the activation of cGMP signaling components. Indeed, the activation of sGC or the inhibition of PDE5 that inhibited the breakdown of cGMP appeared to oppose the negative effect of CFZ on VSM relaxation (FIGS. 4C and 5). The observation of hypophosphorylation of VASP (FIG. 6) draws a similarity to a recent study on endothelium-independent SMC relaxation;^{24,25} it was suggested that the naturally occurring thyroid hormone induced direct vascular relaxation of VSM via the PKG/VASP signaling pathway by increasing the phosphorylation level of VASP.²⁵

To counteract the low phosphorylation levels of VASP, PDE5 inhibitors, including sildenafil and tadalafil, were added to the aortic rings to induce the accumulation of cGMP under endothelial-independent conditions (FIG. 5). This strategy seemed successful as it heightened the phosphorylation level of VASP beyond baseline control. Based on our in vitro results, we postulate that co-administration of PDE5 inhibitors may provide potential cardiovascular protection for at risk patients treated with CFZ. The cardiovascular benefits of PDE5 inhibitors have been recognized; numerous animal studies have demonstrated that PDE5 inhibitors have a protective effect against myocardial ischemia/reperfusion injury, doxorubicin cardiotoxicity, ischemic and diabetic cardiomyopathy, and cardiac hypertrophy.^{26,27} Clinical trials investigating the potential cardiovascular benefits of PDE5 inhibitors are ongoing and may confirm the findings of our ex vivo studies.

Because of the limitation of ex vivo studies, we were unable to evaluate the potential effect of CFZ on the angiotensin pathway in VSM. Clinically, the binding of angiotensin II to the angiotensin II surface receptors of VSM causes the narrowing of blood vessels, which raises blood pressure. Among patients who have high blood pressure, angiotensin-converting enzyme (ACE) inhibitors and angiotensin receptor blockers (ARBs) are commonly given for vasodilation to lower blood pressure.²⁸ Both drugs effectively prevent signal transduction through the angiotensin receptors. Furthermore, ACE inhibitors in combination with β-blockers have been beneficial in chemotherapy-induced cardiotoxicity.^{29,30} It is possible that ACE inhibitors or ARBs, by targeting VSM, can oppose the effect of CFZ; however, further studies are required to clinically validate this theoretical mechanism of action.

Through a well-controlled ex vivo system, the evidence provided here contradicts the belief that CFZ causes endothelial dysfunction. Instead, it would appear that CFZ negatively effects regular vascular relaxation in VSM. The proposed new molecular mechanism of action described here helps to improve our clinical understanding of the potential effect of CFZ on cardiovascular function. Our results demonstrate the protective cardiovascular effect of sGC activators, PDE5 inhibitors, p38 inhibitors, and MAPKAPK-2 inhibitor to find an approach to successfully manage CFZ-induced cardiotoxicity in at-risk patients.

### REFERENCES

1.Dimopoulos M A, Goldschmidt H, Niesvizky R, et al. Carfilzomib or bortezomib in relapsed or refractory multiple myeloma (ENDEAVOR): an interim overall survival analysis of an open-label, randomised, phase 3 trial. Lancet Oncol. 2017; 18:1327-1337.
2. Dimopoulos M A, Moreau P, Palumbo A, et al. Carfilzomib and dexamethasone versus bortezomib and dexamethasone for patients with relapsed or refractory multiple myeloma (ENDEAVOR): a randomised, phase 3, open-label, multicentre study. Lancet Oncol. 2016; 17:27-38.
3. Siegel D S, Dimopoulos M A, Ludwig H, et al. Improvement in Overall Survival With Carfilzomib, Lenalidomide, and Dexamethasone in Patients With Relapsed or Refractory Multiple Myeloma. J Clin Oncol. 2018; 36:728-734.
4. Stewart A K, Rajkumar S V, Dimopoulos M A, et al. Carfilzomib, lenalidomide, and dexamethasone for relapsed multiple myeloma. N Engl J Med. 2015; 372:142-152.
5. Chari A, Stewart A K, Russell S D, et al. Analysis of carfilzomib cardiovascular safety profile across relapsed and/or refractory multiple myeloma clinical trials. BloodAdv. 2018; 2:1633-1644.
6. Dimopoulos M A, Roussou M, Gavriatopoulou M, et al. Cardiac and renal complications of carfilzomib in patients with multiple myeloma. Blood Adv. 2017; 1:449-454.
7. Jakubowiak A J, DeCara J M, Mezzi K. Cardiovascular events during carfilzomib therapy for relapsed myeloma: practical management aspects from two case studies. Hematology. 2017; 22:585-591.
8. Mikhael J. Management of Carfilzomib-Associated Cardiac Adverse Events. Clin Lymphoma Myeloma Leuk. 2016; 16:241-245.
9. Bringhen S, Milan A, D'Agostino M, et al. Prevention, monitoring and treatment of cardiovascular adverse events in myeloma patients receiving carfilzomib A consensus paper by the European Myeloma Network and the Italian Society of Arterial Hypertension. J Intern Med. 2019.
10. Chari A, Hajje D. Case series discussion of cardiac and vascular events following carfilzomib treatment: possible mechanism, screening, and monitoring. BMC Cancer. 2014; 14:915.
11. Rosenthal A, Luthi J, Belohlavek M, et al. Carfilzomib and the cardiorenal system in myeloma: an endothelial effect? Blood Cancer J. 2016; 6:e384.
12. Chen-Scarabelli C, Corsetti G, Pasini E, et al. Spasmogenic Effects of the Proteasome Inhibitor Carfilzomib on Coronary Resistance, Vascular Tone and Reactivity. EBioMedicine. 2017; 21:206-212.
13. Efentakis P, Kremastiotis G, Varela A, et al. Molecular mechanisms of carfilzomib-induced cardiotoxicity in mice and the emerging cardioprotective role of metformin. Blood. 2019; 133:710-723.
14. Kim H R, Graceffa P, Ferron F, et al. Actin polymerization in differentiated vascular smooth muscle cells requires vasodilator-stimulated phosphoprotein. Am J Physiol Cell Physiol. 2010; 298:C559-571.
15. Francis S H, Busch J L, Corbin J D, Sibley D. cGMP-dependent protein kinases and cGMP phosphodiesterases in nitric oxide and cGMP action. Pharmacol Rev. 2010; 62:525-563.
16. Harraz O F, Brett S E, Welsh D G. Nitric oxide suppresses vascular voltage-gated T-type Ca2+ channels through cGMP/PKG signaling Am J Physiol Heart Circ Physiol. 2014; 306:H279-285.
17. Dhein S, Salameh A, Berkels R, Klaus W. Dual mode of action of dihydropyridine calcium antagonists: a role for nitric oxide. Drugs. 1999; 58:397-404.
18. Benz P M, Blume C, Seifert S, et al. Differential VASP phosphorylation controls remodeling of the actin cytoskeleton. J Cell Sci. 2009; 122:3954-3965.
19. Thomas G D, Zhang W, Victor R G. Nitric oxide deficiency as a cause of clinical hypertension: promising new drug targets for refractory hypertension. Jama. 2001; 285:2055-2057.
20. Vasquez-Vivar J, Martasek P, Hogg N, et al. Endothelial nitric oxide synthase-dependent superoxide generation from adriamycin. Biochemistry. 1997; 36:11293-11297.
21. Wolf M B, Baynes J W. The anti-cancer drug, doxorubicin, causes oxidant stress-induced endothelial dysfunction. Biochim Biophys Acta. 2006; 1760:267-271.
22. Kastritis E, Laina A, Gavriatopoulou M, et al. Carfilzomib Induces Acute Endothelial Dysfunction Which Correlates with the Occurrence of Cardiovascular Events [Abstract]. Presented at: Blood, 2018.
23. Quach H, Nguyen K M, Ku M, et al. Characterization of Cardiovascular Adverse Events and B-Type Natriuretic Peptide Levels in Patients with Multiple Myeloma Who Are Treated with Carfilzomib [Abstract]. Presented at: Blood, 2018.
24. Ojamaa K, Klemperer J D, Klein I. Acute effects of thyroid hormone on vascular smooth muscle. Thyroid. 1996; 6:505-512.
25. Samuel S, Zhang K, Tang YD, Gerdes AM, Carrillo-Sepulveda MA. Triiodothyronine Potentiates Vasorelaxation via PKG/VASP Signaling in Vascular Smooth Muscle Cells. Cell Physiol Biochem. 2017; 41:1894-1904.
26. Corinaldesi C, Di Luigi L, Lenzi A, Crescioli C. Phosphodiesterase type 5 inhibitors: back and forward from cardiac indications. J Endocrinol Invest. 2016; 39:143-151.
27. Di Luigi L, Corinaldesi C, Colletti M, et al. Phosphodiesterase Type 5 Inhibitor Sildenafil Decreases the Proinflammatory Chemokine CXCL10 in Human Cardiomyocytes and in Subjects with Diabetic Cardiomyopathy. Inflammation. 2016; 39:1238-1252.
28. Messerli FH, Bangalore S, Bavishi C, Rimoldi SF. Angiotensin-Converting Enzyme Inhibitors in Hypertension: To Use or Not to Use? J Am Coll Cardiol. 2018; 1:1474-1482.
29. Bosch X, Rovira M, Sitges M, et al. Enalapril and carvedilol for preventing chemotherapy-induced left ventricular systolic dysfunction in patients with malignant hemopathies: the OVERCOME trial (preventiOn of left Ventricular dysfunction with Enalapril and caRvedilol in patients submitted to intensive ChemOtherapy for the treatment of Malignant hEmopathies). J Am Coll Cardiol. 2013; 61:2355-2362.
30. Boucek R J, Jr., Steele A, Miracle A, Atkinson J. Effects of angiotensin-converting enzyme inhibitor on delayed-onset doxorubicin-induced cardiotoxicity. Cardiovasc Toxicol. 2003; 3:319-329.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A compound for use in a therapy for treating, inhibiting, reducing, or ameliorating cardiovascular adverse events in a patient being treated with carfilzomib, wherein the compound is a soluble guanylyl cyclase (sGC) activator, P38 inhibitor, MAPKAPK-2 inhibitor, and/or PDE5 inhibitor.

2. The compound for the use of claim 1, wherein the compound is a soluble guanylyl cyclase activator.

3. The compound for the use of claim 1, wherein the compound is a P38 inhibitor and/or MAPKAPK-2 inhibitor.

4. The compound for the use of claim 1, wherein the compound is a PDE5 inhibitor.

5. The compound for the use of claim 2, wherein the therapy further comprises administration of a second compound that is a PDE5 inhibitor, P38 inhibitor, and/or MAPKAPK-2 inhibitor.

6. The compound for the use of claim 3, wherein the therapy further comprises administration of a second compound that is a PDE5 inhibitor or sGC activator.

7. The compound for the use of any one of the preceding claims, wherein the cardiovascular effect is at least one of hypertension, pulmonary hypertension, cardiac failure, ischemic heart disease, or dyspnea.

8. The compound for the use of any one of the preceding claims, wherein the compound is administered prior to, subsequently to, and/or in combination with carfilzomib.

9. The compound for the use of any one of the preceding claims, wherein the compound is administered subsequent to the appearance of the cardiovascular adverse event.

10. The compound for the use of any one of the preceding claims, wherein the compound is a sGC activator chosen from BAY 58-2667 (cinaciguat), BAY 63-2521 (riociguat), BAY 60-2770, S-3448, HMR-1766 (ataciguat), and pharmaceutically acceptable salts thereof

11. The compound for the use of any one of the preceding claims, wherein the compound is a p38 inhibitor chosen from SB202190, SB 203580, neflamapimod, ARRY371797, PF-06802861, PF 07265803, ralimetinib, LY2228820, and pharmaceutically acceptable salts thereof

12. The compound for the use of any one of the preceding claims, wherein the compound is a PDE5 inhibitor chosen from sildenafil, tadalafil, avanafil, vardenafil, phentolamine, yohimbine, L-arginine, and pharmaceutically acceptable salts thereof.

13. The compound for the use of any one of the preceding claims, wherein the patient has increased risk for cardiovascular adverse events or is predisposed for cardiovascular adverse events.

14. The compound for the use of any one of the preceding claims, wherein the patient is a human.

15. The compound for the use of any one of the preceding claims, wherein the patient has multiple myeloma.

16. The compound for the use of any one of the preceding claims, wherein the patient has relapsed or refractory multiple myeloma (RRMM) or newly diagnosed multiple myeloma (NDMM).

17. The compound for the use of any one of the preceding claims, wherein the compound in a pharmaceutical composition comprising the compound and pharmaceutically acceptable excipients.

18. A method of treating, inhibiting, reducing, or ameliorating cardiovascular adverse events in a patient caused by administration of carfilzomib to the patient, said method comprising administering to the patient a pharmaceutically effective amount of at least one compound chosen from soluble guanylyl cyclase (sGC) activator, P38 inhibitor, MAPKAPK-2 inhibitor, and/or PDE5 inhibitor.

19. The method of claim 18, wherein the compound is a soluble guanylyl cyclase (sGC) activator.

20. The method of claim 18, wherein the compound is a p38 inhibitor and/or MAPKAPK-2 inhibitor.

21. The method of claim 18, wherein the compound is a PDE5 inhibitor.

22. The method of claim 19, wherein the method further comprises administration of a second compound that is a PDE5 inhibitor, P38 inhibitor, and/or MAPKAPK-2 inhibitor.

23. The method of claim 20, wherein the method further comprises administration of a PDE5 inhibitor or sGC activator.

24. The method of any one of the preceding claims, wherein said cardiovascular effects is at least one of hypertension, pulmonary hypertension, cardiac failure, ischemic heart disease, or dyspnea.

25. The method of any one of the preceding claims, wherein soluble guanylyl cyclase (sGC) activator, a PDE5 inhibitor, p38 inhibitor, and/or MAPKAPK-2 inhibitor is administered prior to, subsequently to, and/or in combination with carfilzomib.

26. The method of any one of the preceding claims, wherein the compound is administered subsequent to the appearance of the cardiovascular adverse event.

27. The method of any one of claims 18-26, wherein the soluble guanylyl cyclase (sGC) activator is BAY 58-2667 (cinaciguat), BAY 63-2521 (riociguat), BAY 60-2770, S-3448, HMR-1766 (ataciguat), or pharmaceutically acceptable salts thereof.

28. The method of any one of claims 18-27, wherein the method further comprises administering to the patient a PDE5 inhibitor, a P38 inhibitor, and/or MAPKAPK-2 inhibitor.

29. The method of any one of claims 18-28, wherein the PDE5 inhibitor is sildenafil, tadalafil, availed, vardenafil, phentolamine, yohimbine, L-arginine, or pharmaceutically acceptable salts thereof.

30. The method of any one of claims 18-29, wherein the sGC activator is BAY 58-2667 (cinaciguat), BAY 63-2521 (riociguat), BAY 60-2770, S-3448, HMR-1766 (ataciguat), or pharmaceutically acceptable salts thereof

31. The method of any one of claims 18-30, wherein the p38 inhibitor is SB 202190, SB 203580, neflamapimod, ARRY371797, PF-06802861, PF 07265803, ralimetinib, LY2228820, or pharmaceutically acceptable salts thereof

32. The method of any one of claims 18-31, wherein the patient has increased risk for cardiovascular adverse events or is predisposed for cardiovascular adverse events.

33. The method of any one of claims 18-32, wherein the patient is a human

34. The method of any one of claims 18-33, wherein the patient has multiple myeloma.

35. The method of claim 34, wherein the patient has relapsed or refractory multiple myeloma or newly diagnosed multiple myeloma.
